(19) United States
(12) Patent Application Publication
(10) Pub. No.: US 2011/0048841 A1
(43) Pub. Date: Mar. 3, 2011

(54) INTEGRATED STETHOSCOPE AND REFLEX HAMMER AND A METHOD FOR USE

(76) Inventor: William M. Hasbun, Mount Laurel, NJ (US)

(21) Appl. No.: 12/870,675

(22) Filed: Aug. 27, 2010

Related U.S. Application Data
(60) Provisional application No. 61/237,508, filed on Aug. 27, 2009.

(51) Int. Cl. A61B 7/02

(52) U.S. Cl. 181/131

(57) ABSTRACT

A combined diagnostic device for use by medical professionals, which can function as both a stethoscope and a reflex hammer. The combined diagnostic device can function as a stethoscope and a reflex hammer. The diaphragm housing of the stethoscope can be modified so as to act as the head of the reflex hammer and a telescoping handle can connect to and be extend from the diaphragm housing. This design would allow for two instruments to be available to the medical professional without the inconvenience of needing to carry two instruments. The combined diagnostic device would have approximately the same mass and dimensions as a typical stethoscope allowing it to be stored and transported as if it were a typical stethoscope.

### CROSS REFERENCE TO RELATED APPLICATIONS

This application claims benefit to provisional application No. 61/237,508, which is incorporated by reference herein in its entirety.

### FIELD OF THE INVENTION

The present device relates to diagnostic equipment for use by medical professionals. More particularly it relates to stethoscopes and reflex hammers that are used during medical diagnostic procedures. This present device incorporates both of these pieces of equipment into a single medical instrument.

### BACKGROUND

The present device relates to the use of stethoscopes and reflex hammers by medical professionals. Medical diagnostic equipment, such as stethoscopes and reflex hammers, are used by medical professionals to observe the vital signs of patients as part of typical medical examinations. Such examinations can occur as part of routine medical care or during an emergency. Medical professionals should have these important pieces of equipment with them at all times so that they can make quick assessments of a patient's health without extra time wasted looking for misplaced equipment.

A stethoscope is a article of medical equipment that is used to amplify the sounds within a patient's body, which allows the medical professional to evaluate a person's heart, lungs and gastrointestinal tract, among other things. A stethoscope typically comprises a membrane stretched over a bell portion. The membrane contacts the patient's skin and through the bell, the sound is amplified and transferred through flexible tubes to two earpieces, wherein the medical professional can hear the sounds, or feel the vibrations created by the stretched membrane.

A reflex hammer is generally used by a medical professional to assess the nerve responses of a patient. A standard hammer has a hard end piece, sometimes covered with or comprised of rubber, silicone or other similar material, with a rigid shaft that is used to impact specific locations on the body to assess the response created. Some medical professionals have tried to use the bell portion of a typical stethoscope for this purpose with limited success due to the flexible tubing and the awkward angle of the bell portion of the stethoscope with the tubing.

What is needed is a combined diagnostic device comprising both a stethoscope and a reflex hammer in a single device. The device must be able to operate as a standard acoustic stethoscope and a reflex hammer at the option of the user. The ability to move and place the stethoscope head should not be obstructed or impaired by the incorporation of a reflex hammer into the design. Additionally, the reflex hammer portion of the device must be capable of adjusting to and being held firmly at different angles when being used to test different aspects of neurological function. The hammer should be an integral part of the stethoscope and should not increase its size or change its flexibility when the hammer portion is not in use. Moreover, the device must have a rigid handle for when the reflex hammer is being utilized so that the correct force of impact can be applied to the patient. Finally, the combined diagnostic instrument should maintain the same portability and storage characteristics of a standard stethoscope that does not comprise a reflex hammer feature.

### SUMMARY OF THE INVENTION

It is an aspect of the present device to provide an improved combined diagnostic instrument for use by a medical professional, which can act as both a stethoscope and a reflex hammer.

The above aspect can be obtained by a combined diagnostic device comprising: a stethoscope comprising an upper section further comprising a straight tube, tube crotch, and at least one earpiece; and a lower section further comprising a metal body, a cover for the outer edge of the metal body made from a soft material, a handle connected to the metal body, and a diaphragm.

The above aspect can also be obtained by a combined diagnostic device comprising: a stethoscope comprising: an upper section further comprising a straight tube, tube crotch, and at least one earpiece; a lower section further comprising a metal body, a cover for the outer edge of the metal body made from a soft material, a handle connected to the metal body, and a diaphragm; and the handle and metal body also comprising a reflex hammer.

The above aspect can also be obtained by a method for using a combined diagnostic device: providing: a stethoscope comprising: an upper section further comprising a straight tube, tube crotch, and at least one earpiece; and a lower section further comprising a metal body, a cover for the outer edge of the metal body made from a soft material, a handle connected to the metal body, and a diaphragm; the handle and metal body also comprising a reflex hammer; placing the diaphragm into contact with the body of a patient; placing one or more earpieces into the one or more ears of a user and listening for sounds from the patient's body; grasping the handle and striking the patient's body with the metal body to test the patient's reflexes.

### BRIEF DESCRIPTION OF THE DRAWINGS

Further features and advantages of the present device, as well as the structure and operation of various embodiments of the present device, will become apparent and more readily appreciated from the following description of the preferred embodiments, taken in conjunction with the accompanying drawings of which:

FIG. 1/6 is perspective view of a combined diagnostic instrument according to an embodiment;

FIG. 2/6 is an exploded view of the upper section of a combined diagnostic instrument according to an embodiment;

FIG. 3/6 is an exploded view of the lower section of a combined diagnostic device according to an embodiment;

FIG. 4/6 is a perspective view of the reflex hammer assembly section of a combined diagnostic device according to an embodiment;

FIG. 5/6 is a perspective view of a combined diagnostic device with components in a position for its use as a stethoscope according to an embodiment; and

FIG. 6/6 is a perspective view of a combined diagnostic device with its components in a position for its use as a reflex hammer according to an embodiment.

### DETAILED DESCRIPTION

This description of the exemplary embodiments is intended to be read in connection with the accompanying drawings, which are to be considered part of the entire written description.

In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

Reference will now be made in detail to the presently preferred embodiments of the invention, examples of which are illustrated in the accompanying drawings, wherein like reference numerals refer to like elements throughout.

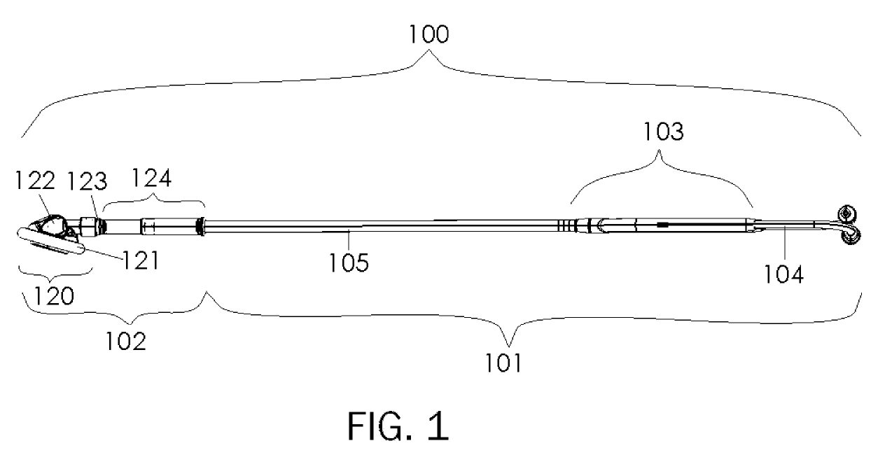

FIG. 1/6 is perspective view of a combined diagnostic instrument 100 according to an embodiment.

Referring to FIG. 1/6, the combined diagnostic instrument 100 can have an upper section 101 and a lower section 102. The upper section 101 can be similar to that of the upper section of a traditional stethoscope and can comprise a standard U-shaped tube crotch 103. The tube crotch 103 can be connected to two earpieces 104 at the top ends of the U and can connect to a straight tube 105 at the middle of the curved portion of the U.

The lower section 102 of the combined diagnostic instrument can comprise both a sound gathering section 120 of the stethoscope, as well as features that allow this section of the device to be used as a reflex hammer. The exterior of the sound gathering section 120 can comprise a large outer rim 121 that can surround the outer edge of a metal body 122. The large outer rim 121 can be made of any suitable material, but silicone is preferable. The metal body 122 can be connected to a threaded connector 123. The threaded connector 123 can attach to a telescoping handle section 124, which can be connected to the straight tube 105.

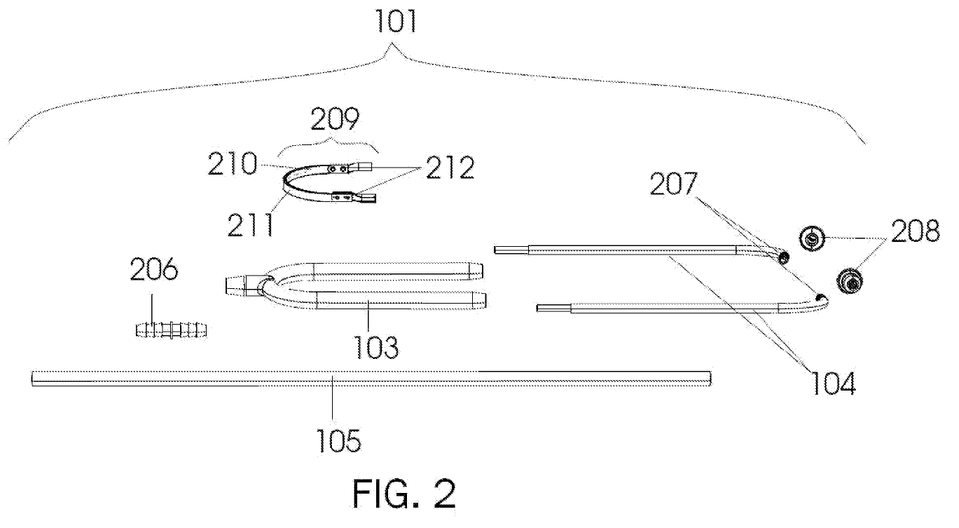

FIG. 2/6 is an exploded view of the upper section 101 of a combined diagnostic device according to an embodiment.

The upper section 101 of the combined diagnostic device can comprise the hearing components for the stethoscope function. The upper section 101 can be connected to the lower section (not shown) through the use of a straight tube 105. The straight tube 105 can be capable of bending into a variety of shapes without kinking, so that the sound waves can still be transmitted through the straight tube 105. Additionally, the straight tube 105 can be capable of being folded and kinked while under pressure, but return to its original shape when the pressure is removed. The straight tube 105 can be made of PVC or another suitable material known to a person skilled in the art, which is capable of allowing sufficient flexibility. The straight tube 105 can be connected to the U-shaped tube crotch 103 through the use of a tube connector 206 which is preferably a hollow piece cylinder with barbs on both ends to securely hold the tubes in place. At the opposite end of the U-shaped tube crotch 103 can connect to two separate ear pieces 104 creating a hollow tube from each ear, through the ear pieces 104, the U-shaped crotch 103, and through the straight tube 105. The earpieces 104 can each comprise a curved bend, which can angle free ends 207 towards each other. The angle can be such that the free ends 207 of each ear piece 104 can be comfortably inserted into the ears of a user. Within the tube crotch 103 can be a tube spring 209. The tube spring 209 can comprise a shorter piece 210 and a longer piece 211, with the two pieces being connected through a tube spring connector 212. The shorter piece 210 can connected to the inside of the longer piece 211, so that the entire tube spring 209 can for a U-shape and it can exert force pulling the free ends 207 towards each other. The tube spring 209 can be made of a metal, plastic, or other material that is sufficiently rigid, but can also be temporarily deformed with the use of force. The tube spring 209 can have spring-like qualities, such that when the tube spring 209 can allow the ear pieces 104 to be pulled away from each other and when the pulling force is removed, the tube spring exerts can bring the earpieces 104 towards each other. This force can help to keep the stethoscope earpieces 104 in the medical professional's ears while it is in use and can keep the stethoscope in a more compact shape when it is not being used.

The free ends 207 can be covered with earpiece caps 208 that can provide comfort to the user. The earpiece caps 208 can be of a shape that provides good acoustic properties by sealing the ear canal, thereby blocking out ambient sounds. It can also be comfortable to rest on a person's neck or be inserted into a person's ears. The tube crotch 103 can be made of PVC or any other suitable material known to a person skilled in the art. The earpiece caps 208 can be made of a silicone material or any other suitable material that is known to a person skilled in the art.

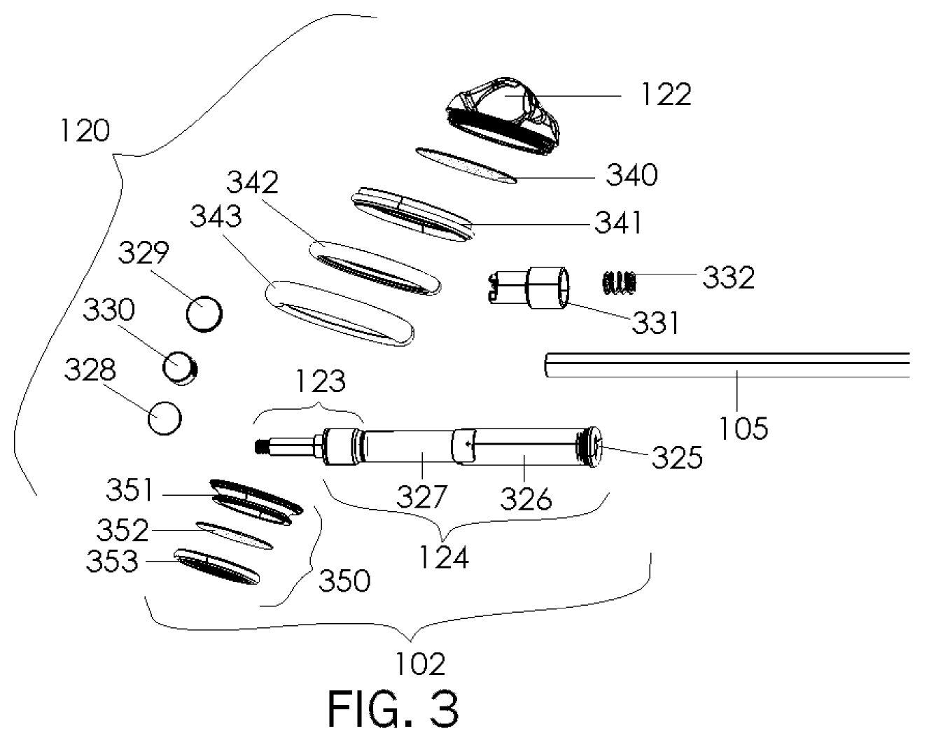

FIG. 3/6 is an exploded view of the lower section 102 of a combined diagnostic device according to an embodiment.

The lower section 102 of the combined diagnostic device can be connected to the upper section (not shown) by the straight tube 105. The straight tube 105 can be secured to the lower portion 102 through the use of a telescoping plug 325. The telescoping plug 325 can secure the straight tube 105 to the outer telescoping handle 326, which can be movably attached to one or more inner telescoping handles 327. Each of one or more inner telescoping handles 327 can be capable of sliding out of the telescoping handle that is one size larger, which can create different lengths of a handle section 124. The inner most telescoping handle can be connected to an inner body plug 328 through the use of a threaded connector 123 and the inner body plug 328 can be secured to the metal body 122 through the use of an outer body plug 329. The inner body plug 328 can be located within an inner rotor 330 so that the handle section 124 and the straight tube 105 are free to rotate about an axis and still be secured to the sound gathering section 120. Surrounding the threaded connector 123 can be a spring loaded detent 331, which can comprise a spring 332 for ease in disengaging a latching mechanism, which can allow the handle to freely rotate to any angle with relation to the metal body 122.

The sound gathering section 120 of the combined diagnostic instrument can be comprised of a metal body 122 that can enclose the inner body plug 328 and inner rotor 330. A large diaphragm 340 can be secured to the metal body 122 through the use of a threaded rim 341. The outer edge of the threaded rim 341 can be covered with a large silicone rim 342, as well as a silicone outer rim 343. The inner circumference of the threaded rim 341 can comprise threading, which can be used to attach a pediatric assembly 350. The pediatric assembly 350 can be utilized when the stethoscope is being used on children or other smaller patients. The smaller sound gathering surface area can also allow the medical professional to listen to target specific internal organs and avoid sounds produced nearby. The pediatric assembly 350 can comprise a small horn 351, which can be connected by threading it onto the inner threading on the threaded rim 341. A small diaphragm 352 can be attached to the small horn 351 through the use of a small silicone rim 353. Although one size of pediatric assembly 350 is depicted, it is understood that any size pediatric assembly 350 can be adapted to attach to the threaded rim 341.

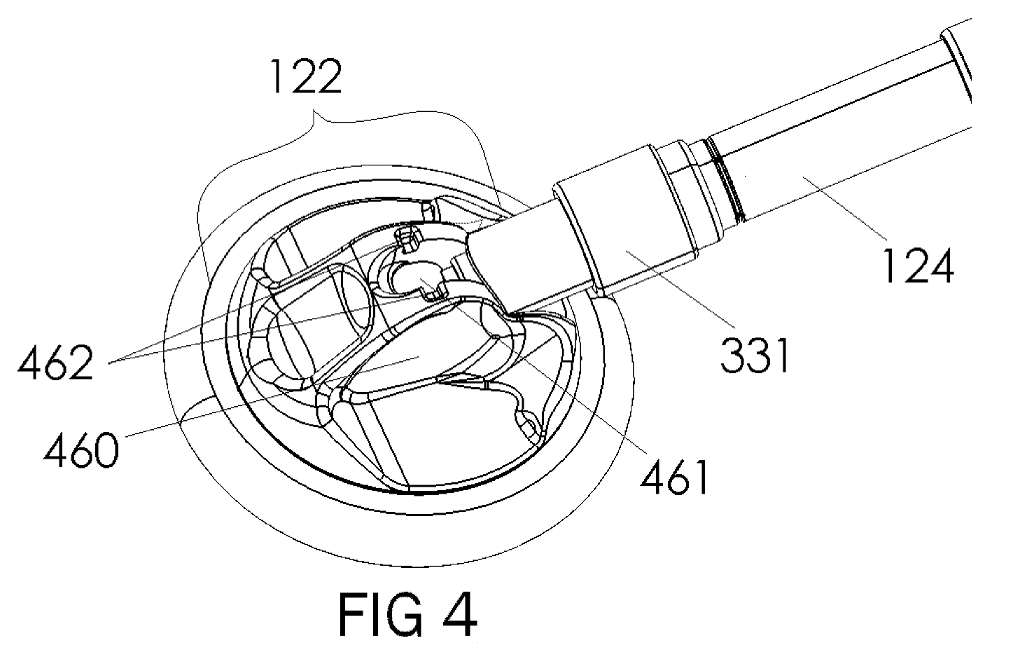

FIG. 4 is a perspective view of the reflex hammer assembly section of a combined diagnostic device.

The metal body 122 can comprise indents 460 on the surface that provide a location for the medical professional to grasp with his or her thumb and finger while using the combined diagnostic device as a stethoscope. The metal body 122 can also comprise a slot 461 for the threaded connector to pass through. The sides of the slot 461 can comprise one or more stops 462. These stops 462 can be used to hold the telescopic handle 124 at certain pre-determined angles. To change the angle of the handle 124 the spring loaded detent 331 can be used to disengage the handle 124 from the slots 462 and the handle 124 can then be freely rotated. The spring loaded detent 331 can be allow to return to its resting state and the handle can snap into the next available slot 462 to be locked at that angle. In an alternative embodiment, the telescopic handle 124 can be locked into position at any angle in relation to the metal body 122.

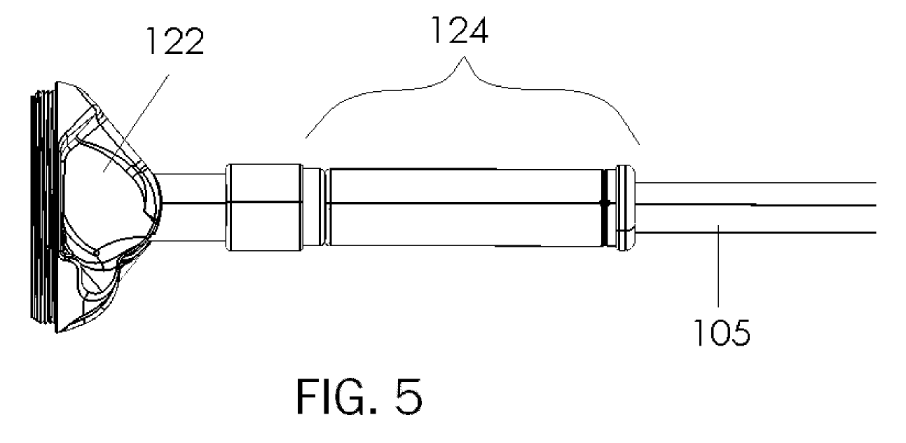

FIG. 5/6 is a perspective view of a combined diagnostic device with components in a preferable position for its use as a stethoscope.

When a medical professional desires to use the combined diagnostic device as a stethoscope, the telescopic handle 124 can be placed in a condensed position. Additionally, the handle 124 can be locked in an angle that holds the handle almost perpendicular to the flat surface of the metal body 122. Although these positions are preferred, it is understood that each medical professional has different opinions and methods for holding a stethoscope. The present combined diagnostic device allows the medical professional to utilize the stethoscope at any angle and handle length combination. The large diaphragm (not pictured) can be placed on the surface of a patient's body and it can pickup and transmit noises from inside the body through the straight tube 105 to the upper section (not pictured) and the user's ears. This angle and handle arrangement is also the preferable storage arrangement because it should be no different than that of traditional stethoscopes, meaning that it should fit around a person's neck or in a pocket, for easy carrying when not in use.

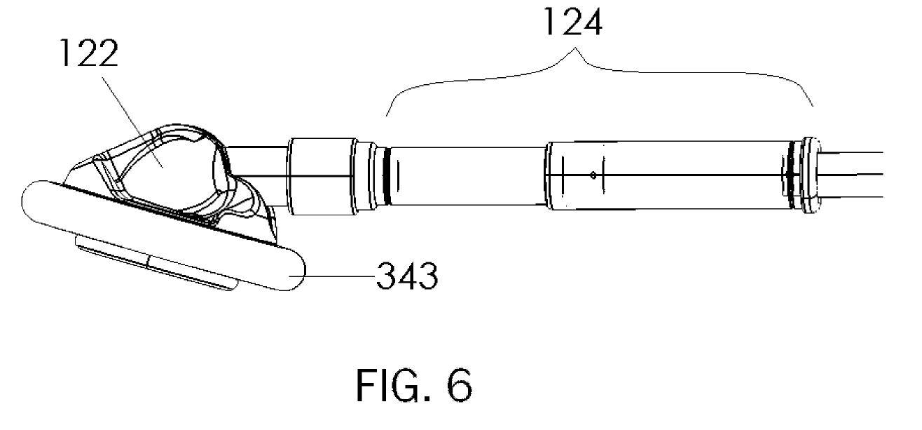

FIG. 6/6 is a perspective view of a combined diagnostic device with its components in the most preferable position for its use as a reflex hammer.

When a medical professional desires to use the combined diagnostic device as a reflex hammer, the telescopic handle 124 can be placed in an extended position. Additionally, the handle 124 can be locked into an angle that holds the handle at the smallest angle with respect to the flat surface of the metal body 122. The silicone outer rim 343 surface provides a cushioned material for striking a person's skin that should not cause pain. The telescopic handle 124 can be grasped by the medical professional and the edge of the metal body 122 covered by the silicone outer rim 343 can be used to strike the desired locations on a patient in the same manner as a traditional reflex hammer to test the neurological response of a patient.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. A combined diagnostic device comprising:
a stethoscope comprising:
an upper section further comprising a straight tube, tube crotch, and at least one earpiece; and
a lower section further comprising a metal body, a cover for the outer edge of the metal body made from a soft material, a handle connected to the metal body, and a diaphragm.

2. The combined diagnostic device as described in claim 1 wherein the handle is a telescoping handle comprising two or more telescoping pieces.

3. The combined diagnostic device as described in claim 1 wherein the straight tube passes through the center of the handle.

4. The combined diagnostic device as described in claim 2 wherein the straight tube passes through the center of the telescoping handle.

5. The combined diagnostic device as described in claim 1 wherein the soft material comprising cover for the outer edge of the metal is silicone.

6. The combined diagnostic device as described in claim 1 wherein the handle can pivot in relation to the metal body.

7. The combined diagnostic device as described in claim 6 wherein the handle can be locked into any number of positions and angles in relation to the metal body using a spring-loaded detent.

8. The combined diagnostic device as described in claim 1 wherein a pediatric assembly, further comprising a small horn can be connected to the metal body.

9. A combined diagnostic device comprising:
a stethoscope comprising:
an upper section further comprising a straight tube, tube crotch, and at least one earpiece;
a lower section further comprising a metal body, a cover for the outer edge of the metal body made from a soft material, a handle connected to the metal body, and a diaphragm; and
the handle and metal body also comprising a reflex hammer.

10. A method for using a combined diagnostic device:
providing:
a stethoscope comprising:
an upper section further comprising a straight tube, tube crotch, and at least one earpiece; and
a lower section further comprising a metal body, a cover for the outer edge of the metal body made from a soft material, a handle connected to the metal body, and a diaphragm;
the handle and metal body also comprising a reflex hammer;
placing the diaphragm into contact with the body of a patient;
placing one or more earpieces into the one or more ears of a user and listening for sounds from the patient's body; and
grasping the handle and striking the patient's body with the metal body to test the patient's reflexes.
